(19) United States
(12) Patent Application Publication
(10) Pub. No.: US 2024/0226078 A9
(43) Pub. Date: Jul. 11, 2024

(54) ENHANCEMENT OF MICROFRACTURE FOR REPAIRING CARTILAGE DEFECTS VIA WNT INHIBITION

(71) Applicant: University of Pittsburgh - Of the Commonwealth System of Higher Education, Pittsburgh, PA (US)

(72) Inventor: Hang Lin, Pittsburgh, PA (US)

(21) Appl. No.: 18/546,644

(22) PCT Filed: Feb. 18, 2022
(86) PCT No.: PCT/US22/16911

§ 371 Date: Aug. 16, 2023

Related U.S. Application Data
(60) Provisional application No. 63/150,613, filed on Feb. 18, 2021.

(51) Int. Cl. A61K 31/444; A61K 9/00; G01N 33/50

(52) U.S. Cl. CPC A61K 31/444; A61K 9/0019; G01N 33/5023

(57) ABSTRACT

A method of performing a microfracture procedure includes creating fractures in subchondral bone and administering a Wnt pathway inhibitor.

### CROSS-REFERENCE TO RELATED APPLICATIONS

This application claims benefit of U.S. Provisional Patent Application Ser. No. 63/150,613, filed Feb. 18, 2021, the disclosure of which is incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Focal articular cartilage injury is a common knee problem that affects approximately 900,000 Americans annually, resulting in greater than 200,000 surgical procedures. Joint injuries incurred during the course of physical labor, training, sport and trauma can cause articular cartilage destruction. Cartilage has very limited self-healing capacity. Moreover, injury is often accompanied by inflammatory and catabolic mediator release. Full-thickness articular cartilage defects thus frequently lead to post-traumatic osteoarthritis (OA), a painful and debilitating joint disease. Timely articular cartilage injury treatment is thus very important to prevent OA onset. Currently, there are several surgical procedures available for knee cartilage defect treatment, including osteochondral allograft transplantation (OAT), autologous chondrocyte implantation (ACI), and microfracture (MFx). In comparison, OAT and ACI each require two expensive and inconvenient surgical procedures, while MFx can be easily performed with a single arthroscopic operation. Given its minimally invasive nature, low cost, and beneficial short-term outcomes, microfracture (MFx), a procedure creating tiny fractures in the subchondral bone, is often considered as the first-line procedure.

MFx is a marrow-stimulating procedure involving subchondral bone perforation, which facilitates bleeding, “super clot” formation, and stem cell migration from bone marrow. These undifferentiated mesenchymal stem cells progressively generate fibrocartilage, a cartilage tissue containing large amounts of collagen type I (COL1). Fibrocartilage is distinctly different from native hyaline cartilage of the healthy articular surface, which is rich in collagen type II (COL2) and aggrecan (AGG), and exclusive of COL1. The fibrocartilage formed after MFx lacks many of the normal hyaline cartilage biochemical and viscoelastic properties, predisposing the repair site to premature degeneration. Consequently, symptoms often reoccur 2-5 years following MFx.

In an effort to promote hyaline-like neotissue formation for long-term repair, as opposed to the short-term benefit of fibrocartilage, several MFx augmentation strategies have been investigated in clinics. For example, scaffolds such as hyaluronic acid and collagen have been applied after MFx in an attempt to create a chondrogenic environment clinically. The platelet-rich plasma was also tested in several studies. However, the utility of such treatments is still unproven and higher-quality evidence indicating inefficacy is still lacking.

In summary, neotissue in the defect site post-MFx is ultimately remodeled into fibrocartilage, which possesses different biochemical and mechanical properties than normal hyaline cartilage, predisposing the repair site to premature degeneration. Consequently, MFx repairs often fail within five years.

It thus remains desirable to develop improved techniques for treatment of cartilage defects.

### SUMMARY

In one aspect, a method of performing a microfracture procedure includes creating fractures in subchondral bone and administering a Wnt pathway inhibitor. In a number of embodiments, the Wnt pathway inhibitor comprises a small molecule drug or siRNA. In general, as used herein small molecule compounds may, for example, have a molecular weight below 1.5 kDa or below 1.0 kDa. The Wnt pathway inhibitor may, for example, include or be XAV939, lorecivivint WNT974, ICG001 IWP 4, ETC-1922159, RXC004, CGX1321, OTSA101-DTPA-90Y, OMP-18R5, OMP-54F28 PRI-724, or SM08502. In a number of embodiments, the Wnt pathway inhibitor includes or is lorecivivint.

The Wnt pathway inhibitor may, for example, be administered after creating the fractures in the subchondral bone. In a number of embodiments, the Wnt pathway inhibitor is administered in the vicinity of the fractures. In a number of embodiments, the Wnt pathway inhibitor is administered via intraarticular injection. The subchondral bone may, for example be subchondral bone of the knee.

In another aspect, a method of screening (including, for example, determining activity and studying activity) at least one molecule for use in suppressing formation of fibrocartilage includes providing a model system including human bone marrow-derived stem cells (hBMSCs) encapsulated within clots derived from human peripheral blood and a chondroinductive factor which induces differentiation into fibrocartilage, applying the at least one molecule to the model system, and monitoring the formation of fibrocartilage in the model system. The at least one molecule for use in suppressing formation of fibrocartilage may, for example, be a Wnt pathway inhibitor may, for example, include or be a small molecule drug or siRNA.

In a number of embodiments, the chondroinductive factor is a synovial fluid chondroinductive factor. The chondroinductive factor may, for example, include or be insulin growth factor-1.

In another aspect, a model system for screening at least one molecule for use in suppressing formation of fibrocartilage includes human bone marrow-derived stem cells (hBMSCs) encapsulated within clots derived from human peripheral blood and a chondroinductive factor which induces differentiation into fibrocartilage. The chondroinductive factor may, for example, be a synovial fluid chondroinductive factor. In a number of embodiments, the chondroinductive factor includes or is insulin growth factor-1.

The present devices, systems, methods and compositions, along with the attributes and attendant advantages thereof, will best be appreciated and understood in view of the following detailed description taken in conjunction with the accompanying drawings.

### BRIEF DESCRIPTION OF THE DRAWINGS

FIG. 1 illustrates a comparison of COL1 gene expression in Control and Wnt inhibition (Optimized) group. A) Reduced COL1 was observed in the Optimized group*, p<0.05. B) Collagen type 1 detection by immunofluorescence. Control group (i-iii); Optimized group (iv-vi). Less COL1 was seen in Optimized group. Bar −50 μm.

FIG. 2 illustrates Western blot analysis for human chondrocytes treated with saline control (CON) and lorecivivint (LRC) for 3 days.

FIG. 3 illustrates post-MFx events including: A) clot formation; B) stem cell migration, C) fibrogenesis, D) chondrogenesis and bone healing, and E) degeneration.

FIG. 4 illustrates an embodiment of an in vitro model hereof.

### DESCRIPTION

The present devices, systems, methods and compositions, along with the attributes and attendant advantages thereof, will best be appreciated and understood in view of the following description taken in conjunction with any accompanying drawings.

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a Wnt inhibitor” includes a plurality of such Wnt inhibitors and equivalents thereof known to those skilled in the art, and so forth, and reference to “the Wnt inhibitor” is a reference to one or more such Wnt inhibitors and equivalents thereof known to those skilled in the art, and so forth. Recitation of ranges of values herein are merely intended to serve as a shorthand method of referring individually to each separate value falling within the range. Unless otherwise indicated herein, and each separate value as well as intermediate ranges are incorporated into the specification as if it were individually recited herein. All methods described herein can be performed in any suitable order unless otherwise indicated herein or otherwise clearly contraindicated by the text.

In a number of embodiments, systems, methods and compositions hereof provide new approaches to microfracture surgery. In a number of embodiments, methods of performing a microfracture procedure hereof include creating fractures in subchondral bone and administering a Wnt pathway inhibitor to the vicinity of the fractures. The timing of the administration of the Wnt inhibitor or pathway inhibitor may vary to be prior to the creation of the fractures (or microfractures), generally at the same time of creation of the fractures, or after creation of the fractures (for example, immediately after or days/weeks/months etc. thereafter). Moreover multiple doses of the Wnt inhibitor may be administered over a period of time. Representative Wnt pathway inhibitors suitable for use herein include, but are not limited to, XAV939 (C₁₄H₁₁F₃N₂OS), lorecivivint WNT974 (C₂₉H₂₄FN₇O), ICG001 (C₃₃H₃₂N₄O₄), IWP 4 (C₂₃H₂₀N₄O₃S₃), ETC-1922159 (C₁₉H₁₇N₇O₃), RXC004, CGX1321, OTSA101-DTPA-90Y (CH₃(CH₂)₁₅CH═CH₂), OMP-18R5 (C₆₃₂₂H₉₇₂₂N₁₆₇₄O₁₉₈₈S₄₆), OMP-54F28, PRI-724 (C₃₃H₃₅N₆O₇P), or SM08502 (C₂₆H₂₂ClFN₈). Wnt signaling and various Wnt inhibitors are, for example, described in Jung, Y S et al., “Wnt signaling in cancer: therapeutic targeting of Wnt signaling beyond β-catenin and the destruction complex”, Experimental and Molecular Medicine, 52, 183-191 (2020), the disclosure of which is incorporated herein by reference.

As set forth above, MFx repairs often fail within five years. The mechanism underlying preferential fibrocartilage formation following MFx has not been fully elucidated. Mirroring the in vivo MFx sequela, in vitro chondrogenesis induction of human bone marrow-derived stem cells (hBMSCs), the major repairing cells after MFx, results in glycosaminoglycan and collagen type II (COL2) production, accompanied by significant collagen type I (COL1) deposition, indicating fibrocartilage formation.

MFx augmentation with stem/progenitor cells by exogenous delivery or endogenous recruitment remain unsuccessful as mechanism to improve regeneration and favor hyaline cartilage differentiation. One theory is that fibrogenesis and chondrogenesis are distinct differentiation programs, and that joint conditions at the time of MFx strongly favor fibrogenesis. Supplementing chondroinductive factors such as bone morphogenetic protein 7 (BMP-7) and fibroblast growth factor 18 (FGF-18) post-MFx increase cartilage matrix deposition in animal models. However, whether these growth factors also significantly suppress fibrosis has not been demonstrated. In addition to fibrosis, poor cartilage matrix deposition is often observed post-MFx, specifically in tissues physically distant from the subchondral bone.

Suppressing Wnt/β-catenin pathway with a tankyrase inhibitor significantly reduces COL1 transcription and deposition without compromising hBMSC chondrogenesis. Wnt/β-catenin inhibition thus has potential to prevent fibrosis while permitting cartilage formation. Most Wnt inhibitors also have the capacity of independently promoting hBMSC chondrogenesis. Without limitation to any mechanism, in the systems, methods and/or compositions hereof MFx may be augmented by promoting chondrogenesis and suppressing fibrosis simultaneously via suppression of the Wnt/β-catenin pathway.

Lorecivivint, a novel Wnt inhibitor that suppresses CDC-like kinase enzyme and dual-specificity tyrosine phosphorylation-regulated kinase enzyme activity, has shown promising results in treating osteoarthritis or OA in, for example, the knee (now in phase 3 clinical trial). Studies indicate that lorecivivint alone is sufficient to initiate hBMSC chondrogenesis with minimal COL1 expression. Lorecivivint injection has been demonstrated to be safe in humans. Lorecivivint's or other Wnt inhibitor's chondroinductive and anti-fibrotic capacity haven't been utilized or studied in the context of MFx. Combining MFx with introducing a Wnt inhibitor such as lorecivivint, however, provides the potential to significantly promote hyaline cartilage formation with reduced fibrosis, thus significantly enhancing MFx chondral repair outcome.

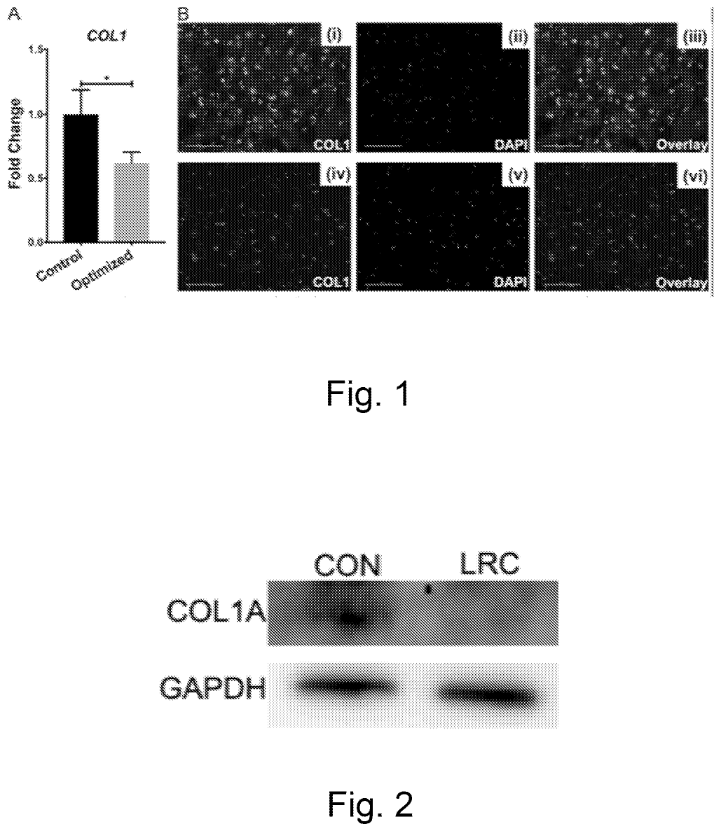

A number of studies of Wnt inhibitors are illustrated in FIGS. 14, 1B and 2. The canonical Wnt/β-catenin signaling pathway plays an important role in fibrotic diseases of other organs. For instance, Wnt/β-catenin signaling is the principal driver in pathological lung and kidney fibrosis. Wnt inhibitors may also suppress fibrogenesis concomitant with chondrogenesis. Upregulated Wnt signaling underlies dermal fibrosis, which can be attenuated by Wnt antagonists. Moreover, Wnt signaling may play an important role in cartilage development. For example, Wnt regulates limb mesenchymal chondrogenesis by modulating N-cadherin-related functions. Additionally, Wnt/β-catenin pathway suppression promotes mesenchymal stem cell (MSC) chondrogenesis or reduces disease progression and severity in OA animal models.

As demonstrated in the studies of FIG. 1A, XAV939, which is a small molecule that selectively inhibits tankyrase and the Wnt pathway, significantly lowers COL1 expression (FIG. 1A) and protein deposition in human bone marrow-derived stem cell (hBMSC)-derived cartilage as compared to controls (see FIG. 1B) without impairing chondrogenesis. In general, as used herein small molecule (therapeutic) compounds may, for example, have a molecular weight below 1.5 kDa or below 1.0 kDa.

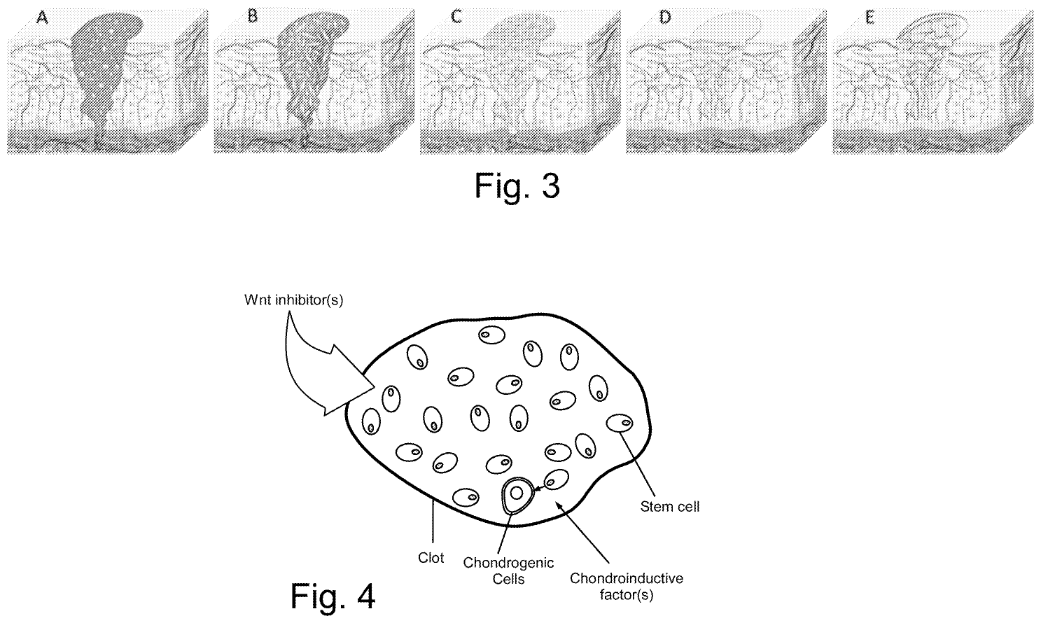

In the methodologies hereof post-MFx intraarticular Wnt inhibitor may, for example, be administered to enhance hyaline cartilage formation, while reducing, minimizing or preventing fibrocartilage formation as seen with current MFx technique. In that regards, FIG. 3 illustrates post-MFx events including: A) clot formation; B) stem cell migration, C) fibrogenesis, D) chondrogenesis and bone healing, and E) degeneration. To facilitate rapid clinical translation, it is desirable to repurpose clinically proven. human-safe Wnt inhibitors. Specifically, the Wnt inhibitor lorecivivint (SM04690), which suppresses CDC-like kinase 2 (CLK2) and dual-specificity tyrosine phosphorylation-regulated kinase 1A (DYRK1A) as well as Wnt/β-catenin signaling, has shown promising results for knee osteoarthritis as discussed above. In addition, studies hereof demonstrated that lorecivivint treatment significantly reduced the COL1 expression in the in vitro-expanded human chondrocytes (FIG. 2). As Wnt inhibitors exhibit dual chondroinductive and anti-fibrotic potential, they have significant potential for use in suppressing post-MFx fibrocartilage formation. The reparative outcome of Wnt- (for example, lorecivivint-) enhanced MFx provides an avenue to achieve long-term hyaline cartilage repair with a minimally invasive and cost-effective procedure, unattainable by current surgical procedures.

Once again, post-MFx use of chondroinductive factors such as bone morphogenetic protein or BMP-7 and FGF-18 has been demonstrated to increase hyaline cartilage formation. Fibroblast growth factor 18 or FGF-18 has also been shown to promote hBMSC. However, the neomatrix has been characterized only by qualitative COL1 and COL2 histochemistry and very limited quantitative data. To address such limitation, and optimize treatments hereof, the regenerated cartilage after Wnt-augmented MFx may be assessed in both quantitative and qualitive manners. Table 1 sets forth a number of studies for characterization of hyaline cartilage and fibrocartilage. In a number of studies, real time-PCR is used in the in vitro studies hereof.

TABLE 1

| | Hyaline Cartilage | Fibrocartilage Specific |
|---|---|---|
| Realtime PCR | COL2, AGA, SOX9, PRG4 | COL1, α-SMA, VSC, FSP1, Tn-C |
| Histology | Safranin O/fast Green, H&E, picosirius red, light microscopy | |
| Immunostaining | COL2, AGG, PRG4 | COL1, 3, -SMA, Tn-C |

| | |
|---|---|
| Others | Magnetic resonance imaging (in vivo), compressive modulus |

COMP: cartilage oligomeric matrix protein; PRG4; proteoglycan 4; α-SMA; α-smooth muscle actin; FSP1; fibroblast-specific protein-1; VSC; vesican; TnC; tenascin-C.

Studies described herein may be used in optimization of Wnt/β-catenin inhibitor introduction in connection with MFx procedures (for example, post-MFx) to reduce fibrosis while enhancing chondrogenesis in the repair of tissue, and, in turn, while promoting stable hyaline cartilage formation for long-term chondral repair. Wnt inhibitor treatment efficacy in promoting hyaline-like cartilage formation in connection with MFx may be studied in, for example, a rat and/or rabbit model. The rabbit model is a more desirable model for the pre-clinical study and modification of MFx. The rabbit model is large enough to simulate clinical surgical treatment while requiring less veterinary expertise and remaining cost effective in comparison to other clinically relevant animal models such as goat and horse. Moreover, the temporal profile of tissue modeling post-MFx has been well studied in rabbits through histology and immunohistochemistry, providing valuable information pertinent to treatment, repair, and endpoint analysis.

The extent of MFx-based repair, augmented by intraarticular Wnt/lorecivivint injection, may, for example, be studied in a lapine iatrogenic microfracture model. Representative Wnt inhibitor administration dosing and scheduling effects may be tested. Repair outcome may, for example, be comprehensively assessed post-surgery based on mechanical in-situ indentation tests, ultra-short echo time-T2* magnetic resonance imaging, and histological/immunohistochemical tissue analysis.

By repurposing the clinically safe lorecivivint (and/or other Wnt inhibitors), methodologies of microfracture/MFx procedures hereof provide a minimally invasive and cost-affordable procedure to achieve robust and long-term chondral defect repair, with the neocartilage possessing native tissue cellular phenotype, biochemical composition, and mechanical properties.

Because intraarticular administration of 0.3 μg lorecivivint dissolved in 50 μl saline (˜12 nM) significantly ameliorates OA progression in rats, a concentration of ˜12 nM may be used in an initial case for rabbit studies. The synovial fluid volume of normal adult rabbit joint is 400-600 μl, and 100-500 μl is typically used for intraarticular injection. An amount of 3 μg lorecivivint may, for example, be dissolved in only 100 μl saline, which will result in a final intraarticular concentration of ˜12 nM after the injected water is absorbed. Concentrations 10-fold higher/lower (1.2 and 120 nM) may be used to confirm that the effects are dose-dependent and/or that negative results are not due to incomplete target coverage. Lorecivivint solubility is ˜5 mM, so desired concentrations can be achieved. Since pharmacokinetic evaluation of a single intraarticular lorecivivint injection demonstrated knee joint residence time>180 days, only one dose is given in a number of studies hereof.

Given its function in chondrogenesis and anti-fibrosis, lorecivivint may, for example, be introduced immediately after MFx. However, Wnt inhibitors have been shown to suppress osteogenesis as well, potentially impairing the bone healing post-MFx and overlying cartilage regeneration. To optimize the lorecivivint treatment time point, one may include additional study groups, testing the effects of delayed treatment. Post-MFx healing process undergoes well characterized phases of repair: fibrous tissue fills the defect in 1-2 weeks, followed by scattered hyaline cartilage formation at 4 weeks concomitant with bone microfracture repair. To determine the best treatment time and balance anti-fibrosis and potential osteogenic suppression, one-time lorecivivint injections may be introduced at, for example, weeks 0, 2, or 4 after MFx surgery.

It is desirable to assess tissue regeneration at different time points to track the whole repair process, but such a procedure is quite expensive. Because the goal is to enhance long-term, stable hyaline cartilage formation, a relatively long-term end point may be used. Regenerated tissue degradation starts as early as 20 weeks after MFx in rabbits. Therefore, based on the results from other studies, 24 weeks may be used to assess MFx's long-term effect.

In a representative example, post-MFx animals may be randomly divided into 10 groups: (1) saline control; (2) 0.3 μg lorecivivint at week 0, (3) 3 μg lorecivivint at week 0, (4) 30 μg lorecivivint at week 0, (5) 0.3 μg lorecivivint at week 2, (6) 3 μg lorecivivint at week 2, (7) 30 μg lorecivivint at week 2, (8) 0.3 μg lorecivivint at week 4, (9) 3 μg lorecivivint at week 4, (10) 30 μg lorecivivint at week 4. Repair outcome are assessed at 24 weeks based on mechanical properties determined by in in situ indentation, T2 magnetic resonance imaging (MRI) may be used to distinguish hyaline cartilage and fibrocartilage (77), and histological/immunohistochemical analysis of tissues may be carried out. MRI analysis may also be performed.

Given the significant clinical studies of lorecivivint and its apparent clinical safety, lorecivivint is thus used as a representative Wnt inhibitor in a number of studies hereof. As described above, the effect of intraarticular lorecivivint injection for MFx-based repair of a surgically generated chondral defect may, for example, be studied and/or optimize in animals such as rats and/or rabbits. Wnt inhibitor (for example, lorecivivint) treatment efficacy in promoting hyaline-like cartilage formation may be studied following MFx in, for example, the rat or rabbit model. In a number of such studies, intraarticular lorecivivint injection occurs subsequent to MFx-based repair of a surgically generated chondral defect in rats. A critical-sized cartilage defect (˜2 mm diameter) may first be made, and then subchondral bone microfracture is immediately performed. After the wound is closed, a saline control or lorecivivint is injected into the vicinity of the MFx treatment (that is, the knee joint cavity). Dosing and timing effects on anti-fibrosis/chondrogenesis and potential osteogenic suppression may be studied. Long-term repair outcome may be comprehensively assessed at one or more defined periods (for example, 12 or 24 weeks) after surgery based on mechanical testing by histological/immunohistochemical tissue analysis. Neocartilage produced with MFx T Wnt inhibitor (for example, lorecivivint) treatment, as compared to MFx alone, has the potential to more closely match hyaline cartilage properties, such as a significantly increased COL2/COL1 ratio.

The cause of post-Mfx fibrocartilage formation remains unclear. An in vitro model hereof may be used to simulate post-MFx tissue remodeling for further study and/or optimization of methodologies hereof Since harvesting human post-MFx clots is not feasible, as one of the initial steps toward human studies/clinical trials, one may simulate such a post-MFx clot by encapsulating stem cells such as hBMSCs within a clot derived from human peripheral blood as described herein. Optimization studies hereof may thus proceed via such a novel in vitro model which simulates post-MFx fibrocartilage formation (see FIG. 3) and assess treatment efficacy. As illustrated schematically in FIG. 4, to mimic the post-MFx clot, stem cells such as human bone marrow-derived stem cells (hBMSCs), the likely progenitor cells contributing to MFx repair, are encapsulated within clots derived from human peripheral blood. hBMSCs/blood clot may be differentiated using, for example, a chondroinductive factor such as insulin growth factor-1 (IGF-1), one of the major synovial fluid chondroinductive factors. The hBMSCs/blood clot may, for example, be induced to differentiate into fibrocartilage upon stimulation by IGF-1 (and/or other chondroinductive factor(s)), at physiological concentration. Application of Wnt inhibitors such lorecivivint in the model allows study of the augmentation of chondrogenesis and/or reduction of fibrosis. The hBMSCs/blood clot is thereby induced to differentiate into fibrocartilage upon such stimulation. Lorecivivint and/or other Wnt inhibitor are added to the clot to study the augmentation of chondrogenesis and reduction of fibrosis.

In a number of embodiments, the model/composition thus includes stem cells such as human bone marrow-derived stem cells or hBMSCs encapsulated within a blood clot derived from human peripheral blood. In a number of embodiments, the methodology includes encapsulating mesenchymal stem cells (human bone marrow-derived stem cells or hBMSCs) within a blood clot derived from human peripheral blood and introducing a chondroinductive factor or factors (for example, including IFG-1) to the encapsulated stem cells.

RNA sequencing (RNA-seq) technology may be used to characterize the global gene expression in the in vitro MFx model, which will not only demonstrate the fibrocartilage formation process and identify new targets to further enhance newly formed cartilage quality but will also reveal the regulation network mediated by a Wnt inhibitor such as lorecivivint. In that regard, one may employ RNA sequencing technology to compare the global gene expression in hBMSCs that are treated with, for example, different concentrations of IGF-1 and/or a Wnt inhibitor such as lorecivivint. Results from transcriptome analysis may be used to demonstrate the molecular mechanism during post-MFx fibrocartilage formation, identifying new targets to further enhance cartilage quality, and also the completed regulation network of lorecivivint as well as other Wnt inhibitors. Results from transcriptome analysis not only uncovers the molecular mechanism during fibrocartilage formation but also reveals the Wnt-inhibitor-mediated regulation network (that is, the factors targets by Wnt inhibitors to regulate hBMSC chondrogenesis).

Thus, in light of findings summarized, for example, in FIGS. 1 and 2, Wnt/β-catenin pathway will be suppressed to reduce post-MFx fibrocartilage formation via Wnt inhibitor such as by repurposing a clinically safe drug, lorecivivint. The in vitro model hereof above may be used to simulate in vivo post-MFx repair, which will allow mechanistic studies as well as understanding of lorecivivint's, and other new treatments', regulation networks. RNA-seq technology, which examines differential gene expression profiles in a much broader dynamic range, may be used to comprehensively assess the new in vitro MFx model, and to identify new drug targets associated with fibrocartilage formation. Moreover, the in vitro model may be used as a high throughput platform for screening MFx-enhancing drug candidates.

Experimental

A. Animal Model. In >6-month-old New Zealand mature rabbits, a full-thickness cartilage defect is created using a 4-mm dermal punch on the weight-bearing medial femoral condyle surface, without injuring subchondral bony structure (78). Using an 18 G needle, 4 MFx holes are created in the defect. The knee is closed and animals are allowed to immediately bear weight. Different lorecivivint doses are intraarticularly administrated at different times. All animals are sacrificed at week 24.

Mechanical assessment. Indentation testing is done to assess the repaired cartilage's mechanical properties (79-81). All tests are performed using a Bose Enduratech ELF 3200 instrument.

MRI. All samples may, for example, be processed in collaboration with the Rangos Research Center Animal Imaging Core.

X-ray microtomography (micro-CT). Subchondral bone formation is, for example, examined with a micro-CT.

Histological assessment. Safranin O and Picosirius Red (observed under polarized light to assess tissue collagen organization) may be used along with the O'Driscoll (MODS) grading system to evaluate the articular cartilage.

Immunohistochemical assessment (IHC). Immunohistochemical tests may be performed following standard protocols for cartilage (COL2, AGG), fibrosis (COL1, COL3), and hypertrophy/bone formation (COL10, MMP13, and RUNX2). The imaging from histology and IHC may be analyzed semi-quantitatively using Image J software. Based on power analysis using the variance from previously published results, 8 samples/group are necessary to achieve statistical significance, providing 95% power to observe effects at α=0.05 when there is a ˜20% difference between groups. Optimizing Wnt/lorecivivint dosage and timing will result in a better reparative outcome defined by: (1) a compressive Young's modulus higher than 50% of surrounding cartilage (84), (2) improved hyaline cartilage formation and integration with significantly reduced fibrosis and hypertrophy as defined by histology, MODS, and MRI, and (3) unimpaired subchondral bone repair.

B. In vitro model—chondrogenesis of hBMSCs/blood clot construct. hBMSCs/blood clots may, for example, be cultured in differentiation medium containing either 0, 10, 20, 50, or 100 ng/ml IGF-1. Lorecivivint may be introduced using the concentrations described above

Preparation of hBMSCs/blood clot construct. Peripheral whole blood, treated with sodium citrate as an anticoagulant, is available from a blood bank. hBMSCs from over 100 male and female patients of varying ages have been isolated that may be used in studies hereof hBMSCs from male and female donors aged 18-50 years may be pooled, representing the major MFx targeted population. P4 hBMSCs are suspended in red blood cell-depleted blood, and clots are formed by mixing with calcium chloride solution.

Analysis of newly formed tissue. Samples may be collected at different time points (Week 0, 1, 2, 4, 6, 8) to evaluate the tissue phenotypes using the methods described in Table 1. Based on power analysis, 6 replicates are required for real time PCR and another 6 replicates are required for histology for each group. Mechanical testing can be performed on the same samples without the need of additional replicates.

RNA-seq. The sequencing platform is Illumina NextSeq with TruSeq Stranded mRNA kit, and the data is paired with the expected read length of 75 bases. RNA-seq data is analyzed using established pipelines, which include: Hisat2 v2.1.0 alignment with Ensembl Grch38 Human reference genome, mapping metrics assessment using QoRTs v1.1.8, quantification using HTSeq v0.9.0, and differential expression analysis using EdgeR (Bioconductor package in R). Pathway analysis is performed using Ingenuity Pathway Analysis. The transcriptome data may, for example, be obtained in a time course experiment to recapitulate the whole regulatory network involved, identifying possible regulators and genes switches responsible.

Fibrocartilage formation may be observed through the in vitro MFx model. After lorecivivint treatment, the following gene expression profiles may be expected: (1) COL2, AGG, SOX9, COMP, PRG4, and COL2/COL1 expression levels higher than untreated group; and (2) COL1, 3, FN, α-SMA expression levels significantly lower compared to the untreated group. COL1 immunostaining is expected to be absent or localized only to the marginal regions of the lorecivivint-treated constructs.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A method of performing a microfracture procedure, comprising:
creating fractures in subchondral bone, and
administering a Wnt pathway inhibitor.

2. The method of claim 1 wherein the Wnt pathway inhibitor comprises a small molecule drug or siRNA.

3. The method of claim 1 wherein the Wnt pathway inhibitor comprises XAV939, lorecivivint WNT974, ICG001, IWP 4, ETC-1922159, RXC004, CGX1321, OTSA101-DTPA-90Y, OMP-18R5, OMP-54F28, PRI-724, or SM08502.

4. The method of claim 1 wherein the Wnt pathway inhibitor comprises lorecivivint.

5. The method of claim 1 wherein the Wnt pathway inhibitor is administered after creating the fractures in the subchondral bone.

6. The method of claim 1 wherein the Wnt pathway inhibitor is administered in the vicinity of the fractures.

7. The method of claim 6 wherein the Wnt pathway inhibitor is administered via intraarticular injection.

8. The method of claim 1 wherein the subchondral bone is subchondral bone of the knee.

9. A method of screening at least one molecule for use in suppressing formation of fibrocartilage, comprising:
providing a model system comprising human bone marrow-derived stem cells (hBMSCs) encapsulated within clots derived from human peripheral blood and a chondroinductive factor which induces differentiation into fibrocartilage,
applying the at least one molecule to the model system, and
monitoring the formation of fibrocartilage in the model system.

10. The method of claim 9 wherein the at least one molecule for use in suppressing formation of fibrocartilage is a Wnt pathway inhibitor which comprises a small molecule drug or siRNA.

11. The method of claim 9 wherein the chondroinductive factor is a synovial fluid chondroinductive factor.

12. The method of claim 9 wherein the chondroinductive factor is insulin growth factor-1.

13. A model system for screening at least one molecule for use in suppressing formation of fibrocartilage, comprising: human bone marrow-derived stem cells (hBMSCs) encapsulated within clots derived from human peripheral blood and a chondroinductive factor which induces differentiation into fibrocartilage,

14. The model of claim 13 wherein the chondroinductive factor is a synovial fluid chondroinductive factor.

15. The model of claim 13 wherein the chondroinductive factor is insulin growth factor-1.
